(19) United States
(12) Patent Application Publication
(10) Pub. No.: US 2020/0378980 A1
(43) Pub. Date: Dec. 3, 2020

(54) TEST PIECE FOR DETECTION OF KETONE BODIES IN URINE

(71) Applicant: SYSMEX CORPORATION, Kobe-shi (JP)

(72) Inventors: Akiko ISHIDA, Taito-ku (JP); Kazuo Ohata, Taito-ku (JP)

(73) Assignee: SYSMEX CORPORATION, Kobe-shi, JAPAN

(21) Appl. No.: 16/997,153

(22) Filed: Aug. 19, 2020

Related U.S. Application Data
(63) Continuation of application No. PCT/JP2019/006596, filed on Feb. 21, 2019.

(30) Foreign Application Priority Data
Feb. 23, 2018 (JP) 2018-030561

(51) Int. Cl. G01N 33/64; G01N 21/78

(52) U.S. Cl. CPC G01N 33/64; G01N 21/78

(57) ABSTRACT

In measurement of ketone bodies in urine, false-positives due to a drug having a thiol group are inhibited. False-positives due to a drug having a thiol group are inhibited by adding an oxidizing agent to a test piece for detection of ketone bodies in urine.

### CROSS REFERENCE TO RELATED APPLICATIONS

This application is a continuation of International Application PCT/JP2019/006596 filed on Feb. 21, 2019, which claims benefit of Japanese patent application JP 2018-030561 filed on Feb. 23, 2018, both of which are incorporated herein by reference in their entireties.

### BACKGROUND OF THE INVENTION

### Field of the Invention

The present invention relates to inhibition of non-specific reactions in measurement of ketone bodies in urine.

### Description of the Background Art

Detection of various components in urine is used for diagnoses of various diseases, in particular, renal disease, urinary tract disease, and diabetes. Collection of a urine specimen hardly causes any burden to a subject, and thus, a urine test is not only performed for patients of hospitals, but also always performed in health examinations.

In general, a urine test strip to be used in a test of a urine specimen has a form in which a plurality of test pieces capable of detecting various components in urine through color reactions are attached to a synthetic resin support body such as PET. The urine test strip is immersed in a urine specimen collected in a paper cup or the like and then is taken out, colorations of test piece portions are visually confirmed, and presence or absence of various components is qualitatively (positive or negative) determined. When the test is performed using a fully automatic analyzer, a urine specimen is dropped on a urine test strip, colorations are optically detected, and the results are compared to a calibration curve, whereby concentrations of various components are calculated.

Examples of items detectable by a urine test strip include glucose, protein, occult blood, and ketone bodies. Other than these, creatinine can also be measured, and the amount of urine protein per day can also be calculated at any time from the protein concentration in urine.

Ketone bodies are a generic term of acetone, acetoacetic acid, and β-hydroxybutyric acid, and usually, ketone bodies are not detected in urine of healthy persons. However, it is known that ketone bodies in urine become positive in the cases of diabetes, hyperthyroidism, and the like.

The detection principle of ketone bodies in urine uses a phenomenon that sodium nitroprusside reacts with acetone or acetoacetic acid to exhibit a purple color. However, sodium nitroprusside also similarly reacts with a compound having a thiol group and exhibits a purple color. That is, when a subject is taking a drug that contains a compound having a thiol group, the drug is discharged into urine and reacts with sodium nitroprusside, whereby the test result of ketone bodies in urine becomes false-positive.

Captopril (antihypertensive drug) and bucillamine (antirheumatic drug) are known as drugs having a thiol group that causes a false-positive of ketone bodies in urine. In particular, since bucillamine could cause proteinuria as a side effect, urine qualitative tests are frequently performed for bucillamine-treated patients. Thus, a false-positive of ketone bodies in urine poses a problem.

### SUMMARY OF THE INVENTION

A problem to be solved by the present invention is to inhibit false-positives due to a drug having a thiol group in measurement of ketone bodies in urine.

In order to solve the above problem, the present inventors conducted thorough research and completed the invention indicated below.

The present invention provides a test piece for detection of ketone bodies in urine and a false-positive inhibition method indicated below.
(1) A test piece for detection of ketone bodies in urine, the test piece containing an oxidizing agent.
(2) The test piece for detection of ketone bodies in urine according to (1), wherein the oxidizing agent is an oxidizing agent for inhibiting a false-positive.
(3) The test piece for detection of ketone bodies in urine according to (1) or (2), wherein the oxidizing agent is potassium iodate.
(4) A false-positive inhibition method comprising adding an oxidizing agent in a test piece for detection of ketone bodies in urine.
(5) The false-positive inhibition method according to (4), wherein the oxidizing agent is potassium iodate.

Since the test piece for detection of ketone bodies in urine of the present invention contains the oxidizing agent, even when a subject of a urine test is taking a drug having a thiol group, a result of the test can be inhibited from becoming false-positive.

These and other objects, features, aspects and advantages of the present invention will become more apparent from the following detailed description of the present invention when taken in conjunction with the accompanying drawings.

### DESCRIPTION OF THE PREFERRED EMBODIMENTS

A test piece for detection of ketone bodies in urine of the present invention is created as a test piece that contains ketone bodies detection reagent, as described in detail below. Further, a urine test strip for a multi-item test may be formed by providing a test piece for another detection target item on a support body. Alternatively, a test strip for a single item for detecting only ketone bodies in urine may be formed.

The test piece for detection of ketone bodies in urine of the present invention comprises a reagent composition that contains sodium nitroprusside and an oxidizing agent. In the test piece for detection of ketone bodies in urine of the present invention, the oxidizing agent can inhibit sodium nitroprusside from: reacting with a compound having a thiol group; and non-specifically exhibiting coloration to cause a false-positive. The oxidizing agent is not limited in particular, and an alkali metal salt of iodic acid or potassium ferricyanide can be used. Out of these oxidizing agents, potassium iodate is preferable.

The test piece for detection of ketone bodies in urine of the present invention is created by: preparing a solution that contains sodium nitroprusside and an oxidizing agent; causing an absorbent carrier to be impregnated with the solution; and drying the resultant absorbent carrier. The concentration of sodium nitroprusside in the solution is preferably 0.5% to 1.5%. The concentration of the oxidizing agent in the solution is preferably 0.25% to 0.75%.

As the reagent composition to be carried by the test piece, a solubilizer, a sensitizer, a wetting agent, a buffer agent, etc., may be used, in addition to sodium nitroprusside and the oxidizing agent.

Filter paper is often used as the absorbent carrier to be impregnated with the reagent composition. However, cotton, nonwoven fabric, glass fiber, or the like can also be used. Further, an organic polymer such as gelatin or synthetic resin can also be caused to contain the reagent composition.

The test piece for detection of ketone bodies in urine manufactured in this manner is attached to a support body. The test piece for detection of ketone bodies in urine may be attached to the support body together with a test piece that detects another detection target item. For example, a support body in the form of a plastic piece or sheet made from polyvinyl chloride, polyethylene terephthalate, polypropylene, polystyrene, polyester, or the like can be used as the support body.

A test strip obtained in this manner is immersed in a liquid sample that contains urine, and is immediately taken out. Then, coloration of the test piece after a certain time is compared, with eyes, against a previously-created standard color table, whereby a qualitative or semi-quantitative test of the sample is performed. Alternatively, a quantitative test can also be performed by optically measuring reflectance or the like by use of a measurement apparatus, and obtaining a concentration with reference to a calibration curve.

### EXAMPLE

### Example 1

### Test Piece, for Detection of Ketone Bodies in Urine, Containing Oxidizing Agent

A solution, for detection of ketone bodies in urine, that contains 0.8% sodium nitroprusside, 0.4% potassium iodate, and glycine buffer solution (pH 9.0) was prepared. Then, filter paper was caused to be impregnated with the solution and then dried, whereby a test piece for detection of ketone bodies in urine was produced.

### Comparative Example 1

### Test Piece for Detection of Ketone Bodies in Urine, for Control

A test piece for detection of ketone bodies in urine, for control, was created in the same manner as that for Example 1, except that the control test piece did not contain potassium iodate.

### Example 2

200 urine specimens were measured, using the test pieces, for detection of ketone bodies in urine, that were created in Example 1 and Comparative Example 1. There were 25 specimens that were negative with the test pieces containing the oxidizing agent but that were positive with the control test pieces. With respect to each of these 25 specimens, ketone bodies measurement result by an enzyme method indicated 0.0 mg/dL (not greater than detection sensitivity). All of these 25 specimens were urine of rheumatoid arthritis patients who were taking bucillamine.

Since the test piece for detection of ketone bodies in urine of the present invention contains the oxidizing agent, the test piece for detection of ketone bodies in urine can effectively inhibit non-specific reactions due to a compound having a thiol group. Therefore, even when a subject of a urine test is taking a drug having a thiol group, the result of a test of ketone bodies in urine can be inhibited from becoming false-positive.

While the invention has been described in detail, the foregoing description is in all aspects illustrative and not restrictive. It will be understood that numerous other modifications and variations can be devised without departing from the scope of the invention.

What is claimed is:

1. A test piece for detection of ketone bodies in urine, the test piece comprising
an oxidizing agent.

2. The test piece for detection of ketone bodies in urine according to claim 1, wherein
the test piece comprises sodium nitroprusside.

3. The test piece for detection of ketone bodies in urine according to claim 1, wherein
the oxidizing agent is an oxidizing agent for inhibiting a false-positive.

4. The test piece for detection of ketone bodies in urine according to claim 1, wherein
the oxidizing agent oxidize a drug having a thiol group that causes a false-positive.

5. The test piece for detection of ketone bodies in urine according to claim 4, wherein
the drug having a thiol group comprises bucillamine and captopril.

6. The test piece for detection of ketone bodies in urine according to claim 1, wherein the oxidizing agent is potassium iodate.

7. The test piece for detection of ketone bodies in urine according to claim 1, wherein the test piece is attached to a support body.

8. The test piece for detection of ketone bodies in urine according to claim 7, wherein the support body is a plastic piece or sheet whose material is selected from the group comprising of polyvinyl chloride, polyethylene terephthalate, polypropylene, polystyrene or polyester.

9. A false-positive inhibition method comprising:
adding an oxidizing agent in a test piece for detection of ketone bodies in urine.

10. The false-positive inhibition method according to claim 9, wherein the oxidizing agent oxidize a drug having a thiol group that causes a false-positive.

11. The drug according to claim 10, wherein the drug having a thiol group comprises bucillamine and captopril.

12. The false-positive inhibition method according to claim 9, wherein the oxidizing agent is potassium iodate.

13. The false-positive inhibition method according to claim 9, further comprising
detecting coloration of the test used in the adding step.

14. A method for detection of ketone bodies in urine comprising:
contacting a test piece comprising sodium nitroprusside and an oxidizing agent with a urine specimen; and,
detecting ketone bodies in the urine specimen with the test piece used in the contacting step.

15. The method for detection of ketone bodies according to claim 14, wherein the oxidizing agent oxidize a drug having a thiol group that causes a false-positive.

16. The method for detection of ketone bodies according to claim 15, wherein the drug having a thiol group comprises bucillamine and captopril.

17. The method for detection of ketone bodies according to claim 14, wherein the oxidizing agent is potassium iodate.

18. The method for detection of ketone bodies according to claim 14, wherein the detecting step is based on detecting a coloration of the test piece.

19. The method for detection of ketone bodies according to claim 18, wherein the detecting step comprises comparing the coloration of the test piece with a standard color table.

20. The method for detection of ketone bodies according to claim 14, wherein the detecting step comprises detecting, by use of a measurement apparatus, a coloration of the test piece; and obtaining a concentration with reference to calibration curve based on the coloration detected by the measurement apparatus.
